(19) United States
(12) Patent Application Publication
(10) Pub. No.: US 2018/0369499 A1
(43) Pub. Date: Dec. 27, 2018

(54) SYRINGES WITH RETRACTABLE NEEDLE

(71) Applicant: IINJEC TECHNOLOGIES INC. / LES TECHNOLOGIES IINJEC INC., Montrea (CA)

(72) Inventors: Catherine Reisenburg MOLSON, Montreal (CA); James JACKSON, Victoria (CA); Scott PHILLIPS, Victoria (CA); Timothy CUMMINGS, Victoria (CA)

(21) Appl. No.: 16/063,163

(22) PCT Filed: Dec. 15, 2016
(86) PCT No.: PCT/CA2016/051481

§ 371 Date: Jun. 15, 2018

Related U.S. Application Data
(60) Provisional application No. 62/267,351, filed on Dec. 15, 2015.

(51) Int. Cl. A61M 5/32; A61M 5/28; A61M 5/315; A61M 5/34; A61M 5/42

(52) U.S. Cl. CPC A61M 5/3234; A61M 5/28; A61M 5/46; A61M 5/34; A61M 5/425; A61M 5/315

(57) ABSTRACT

The syringe can have a plunger body and a plunger seal both independently slidingly engaged within a cavity of a barrel for movement along a common longitudinal axis, and a needle anchored to the plunger body and extending across the plunger seal, the needle having an inlet aperture allowing fluid flow communication into an internal conduit of the needle, the barrel having a reservoir for receiving a medical fluid, the reservoir extending between the plunger seal and a bottom, the bottom being closed by a septum.

### BACKGROUND

Syringes with retractable needles can have the advantage of moving the needle out from exposure once the injection has been performed, potentially presenting a particular interest for disposal, or otherwise for use by persons who do not have a profound medical training. International patent application publication WO2014096957 presents various embodiments of syringes with retractable needles.

While existing technology was found satisfactory to a certain extent, there remained room for improvement.

### SUMMARY

In accordance with one aspect, there is provided a syringe having a plunger body and a plunger seal both independently slidingly engaged within a cavity of a barrel for movement along a common longitudinal axis, and a needle anchored to the plunger body and extending across the plunger seal, the needle having an inlet aperture allowing fluid flow communication into an internal conduit of the needle, the barrel having a reservoir for receiving a medical fluid, the reservoir extending between the plunger seal and a bottom, the bottom being closed by a septum, wherein to proceed with injection, the plunger body is slid toward the plunger seal along a penetration span, during which movement the needle punctures the septum and protrudes from the bottom without injecting medical fluid, until the plunger body engages the plunger seal, after which the plunger body and the plunger seal are collectively moved to inject the medical fluid until the plunger seal reaches the bottom.

In accordance with another aspect, there is provided a syringe having a plunger having a plunger seal slidingly engaged within a cavity of a barrel, and a needle anchored to the plunger, the barrel having a reservoir for receiving a medical fluid, the reservoir extending between the plunger seal and a bottom, the bottom being closed by a septum, the needle having a lateral aperture allowing fluid flow communication between the reservoir and an internal conduit of the needle, wherein to proceed with injection, the plunger seal and the anchored needle are collectively moved along an axis of the syringe toward the bottom to inject the medical fluid until the plunger seal reaches the bottom, wherein the lateral aperture of the needle extends along a given axial length immediately below the plunger seal in a manner remain exposed to the reservoir until the plunger seal has reached the bottom.

In accordance with another aspect, there is provided a syringe having a plunger anchor to which a needle is anchored and a plunger seal both slidingly engaged within a cavity of a barrel, and a plunger actuator having at least two elastic arms slidingly engaged around the barrel, the arms being biased to a radially inward position and prevented by the barrel, the barrel having a septum seal at a bottom and an open top, wherein to proceed with injection, the plunger actuator is pulled upwardly until the arms are freed from the barrel, at which points the arms are freed and move to the radially inward position, and the plunger actuator can be pushed downwardly to engage the arms into the barrel via the open top, until they engage the plunger anchor and become operable to push the needle across the septum.

Many further features and combinations thereof concerning the present improvements will appear to those skilled in the art following a reading of the instant disclosure.

### DESCRIPTION OF THE FIGURES

In the figures,

FIGS. 1A, 1B and 10 is a sequence of three cross-sectional views of a first embodiment of a syringe, in which the syringe is shown in different configurations;

FIGS. 2A and 2B show a sequence of 7 cross-sectional views of a second embodiment of a syringe, showing sequential configurations of the syringe during operation;

FIG. 3A is a side elevation view of a third embodiment of a syringe, with FIG. 3B being a cross-section thereof taken along cross-section lines 3B-3B of FIG. 3A;

FIG. 3C is an oblique view of the syringe of FIG. 3A, shown with a stop;

FIG. 3D is a fragmented, oblique cross-sectional view of the syringe of FIG. 3A shown at a later configuration of use;

FIG. 4 is a sequence of 4 cross-sectional views of a fourth embodiment of a syringe, showing sequential configurations of the syringe during operation;

FIGS. 5A and 5B show a sequence of 8 cross-sectional views of a fifth embodiment of a syringe, showing sequential configurations of the syringe during operation;

FIG. 5C is an oblique view of a component of the syringe of FIGS. 5A and 5B.

### DETAILED DESCRIPTION

The figures show syringes with a confined needle, a reservoir with a prefilled medical fluid, and a plunger to which the needle is anchored. The plunger is in two parts: a body and a seal.

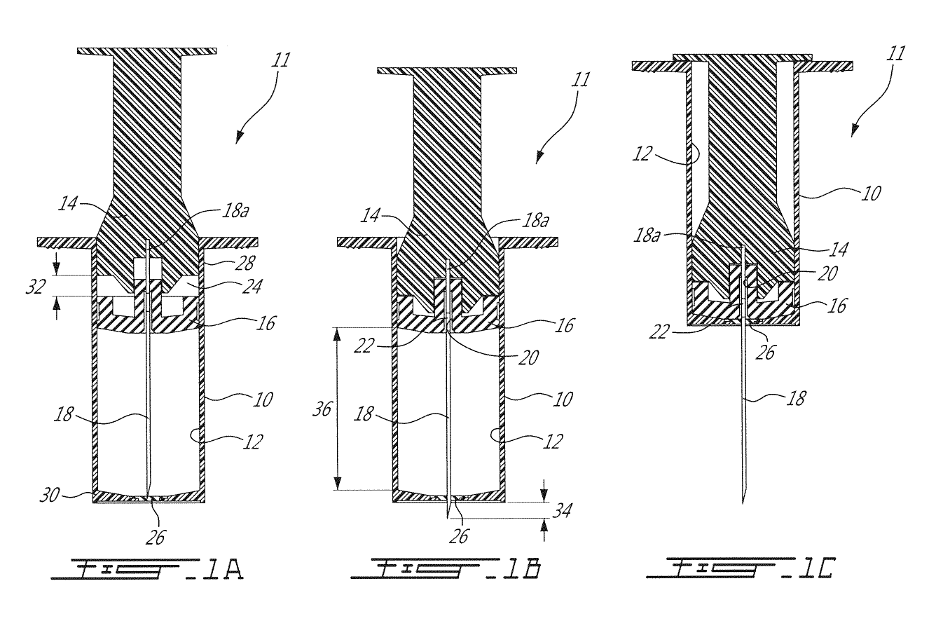

A first embodiment of a syringe is shown in FIGS. 1A to 10. The syringe 11 has a barrel 10 defining a reservoir 12 containing the medical fluid. A plunger body 14 and a plunger seal 16 can independently slide back and forth along the longitudinal axis of the barrel 10. A needle 18 is anchored in a head of the plunger body 14, at 18a and passes through the plunger seal 16 in a passage 20 defined in the plunger seal 16, along the longitudinal axis of displacement. The needle 18 has an inlet 22 as shown. More specifically, the needle 18 is provided in the form of a straight metal tube having a first end anchored in the plunger body 14, the straight metal tube projecting from the plunger body 14 along the longitudinal axis of displacement and leading to a free end having a beveled tip. The inlet 22 is defined transversally across a portion of the metal tube, and allows fluid flow communication between the outside of the metal tube and an internal conduit inside the metal tube leading to the free end for medical fluid injection.

FIG. 1A shows the syringe 11 in its initial configuration, prior to injection. In the initial configuration, the plunger seal 16 is positioned at an intermediary position along the longitudinal axis of displacement whereas the head of the plunger body 14 is positioned at a first end 28 of the barrel 10. A spacing 24 is provided between the plunger body 14 and the plunger seal 16. The barrel 10 has a second end 30 opposite the first end 28. A septum 26 initially closes the second end, and the medical fluid is trapped between the plunger seal 16, the peripheral wall of the barrel 10, and the septum 26. The plunger seal 16 has a cylindrical recess surrounding the needle 18 and the inlet 22. This cylindrical recess, as well as the internal needle conduit, are also filled with medical fluid in the initial position.

To proceed to injection, the second end 30 of the barrel 10, and the septum 26, are positioned against the skin of the patient. The plunger body 14 is then activated to move along the longitudinal axis of displacement, moving the anchored needle 18 with it. The plunger body 14 will first advance along a penetration span 32 corresponding to the longitudinal length of the spacing 24, and the free end of the needle 18 will pierce the septum 26 and penetrate into the body of the patient. During this movement, medical fluid is free to move into the cylindrical recess, across the inlet 22, and be injected into the patient. However, the volume of the reservoir 22 remains the same during this movement, and fluid is not pushed across the needle aperture 22 and into the patient until the plunger body 14 engages the plunger seal 16 in the configuration shown in FIG. 1B. This can allow the needle tip to reach a given penetration distance 34 beneath the skin of the patient prior to beginning injection.

In an alternate embodiment, the internal needle conduit is filled with medical fluid in the initial position, but the plunger seal 16 does not have a cylindrical recess and rather abuts against the needle inlet 22 and an additional portion of the needle under the needle inlet, in a manner that the needle inlet 22 is sealed by the plunger seal 16 and remains sealed by the plunger seal as the plunger body 14 is moved along the penetration distance. The needle inlet 22 only becomes exposed to the reservoir 12 once the plunger body 14 has been moved along the penetration span 32 and needle inlet 22 has crossed the plunger seal 16.

Referring now to FIGS. 1B and 10, once the plunger body 14 has reached and abuts against the plunger seal 16, further movement of the plunger body 14 will move not only the plunger body 14, but also the plunger seal 16, which, in turn, confines the volume of the reservoir 12. The volume-confining action entrains movement of the medical fluid across the needle inlet 22, along the needle conduit, and out the needle tip into the patient, as the plunger body 14 and plunger seal 16 are collectively moved along an injection span 36 from the position shown in FIG. 1B to the position shown in FIG. 10. In the position shown in FIG. 10, the medical fluid has been completely injected into the patient's body.

It will be noted here that in the position shown in FIG. 10, the volume-confining face of the plunger seal 16 closely matches the shape of the bottom of the barrel, and the position of the aperture 22 can be adjusted in a manner that the aperture 22 remains exposed to the medical fluid until the very end of the movement (the point where the plunger seal 16 meets the septum 26 and the bottom of the barrel 10). Accordingly, the volume of medical fluid which remains undispensed after the injection operation can be minimized.

From that position, the movement of the plunger body 14 is reversed, pulling the needle 18 back into the reservoir 12 (not shown). During the reversed movement, the friction between the plunger seal 16 and the barrel 10 is greater than the friction between the plunger seal 16 and the needle 18, and the plunger seal 16 will typically remain in its fully deployed position as the needle 18 is retracted. Once the needle aperture 22 has moved across the plunger seal 16 and is exposed to the atmosphere, the internal needle conduit remains at atmospheric pressure.

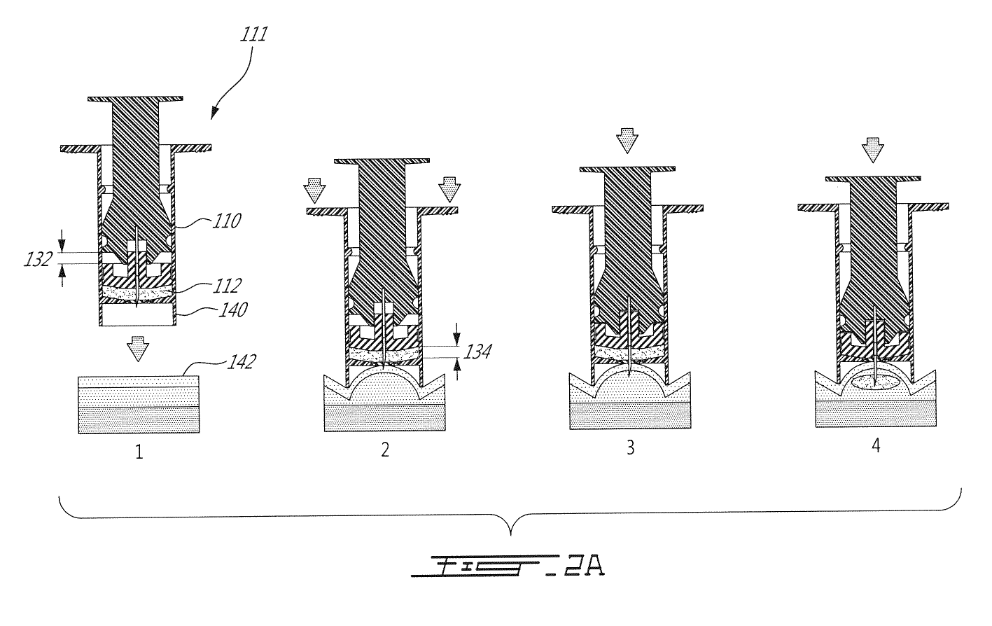

FIGS. 2A and 2B show a similar embodiment along successive operation steps 1 to 7. Two differences with the embodiment of FIG. 1A to 10 will be discussed. A first one of these differences is that the bottom of the barrel 110 has a projecting neck 140 which projects peripherally opposite the reservoir 112. The projecting neck 140 engages the skin 142 of the patient as shown in step 2, which can cause a puckering, or bulging, of the skin 142 as shown. This puckering or bulging of the skin 142 can be particularly useful in embodiments where the syringe 111 is designed for injection at sub-cutaneous depths. A second one of these differences is the presence of a rib 144 protruding transversally inwardly from the barrel 110 near the upper end 128, and a corresponding female feature 146, shown here in the form of a groove or channel, provided in the head of the plunger body 114. When the plunger body 114 is fully retracted, such as shown at step 7, the rib 144 engages the female peripheral channel 146 provided around the head of the plunger body 114, effectively snapping the plunger body 114 in the fully retracted position.

It will be noted that the reservoir 112 generally has a cylindrical shape in this embodiment. The exact volume of a specific design which departs from a cylindrical shape can be calculated using a computer assisted drawing software, for instance. Nonetheless, to evaluate the general principles, we will look into an example cylindrical shape. A cylindrical volume can be calculated by the equation V=πr²h where r is the radius and h is the height. In this example, it will be understood that the reservoir can be designed in a manner for the injecting action of a given volume of medical fluid to be confined within a given height span of the reservoir. Indeed, this can be achieved by adapting the radius r of the reservoir accordingly. A reservoir having a larger radius, for a given volume, can be used for sub-cutaneous injection, whereas a reservoir having a smaller radius and a greater height, for a given volume, can be used for intramuscular injection, for instance. The penetration span 132 can be adjusted independently from the injection span 134 as can be understood from the above. The design shown in FIGS. 2A and 2B is adapted for sub-cutaneous injection.

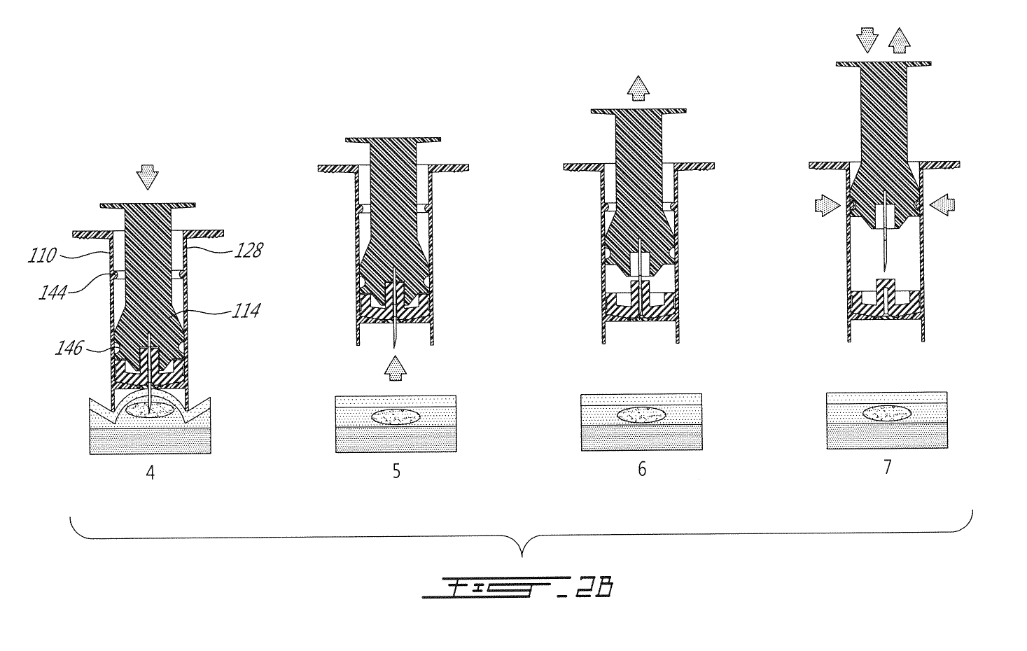

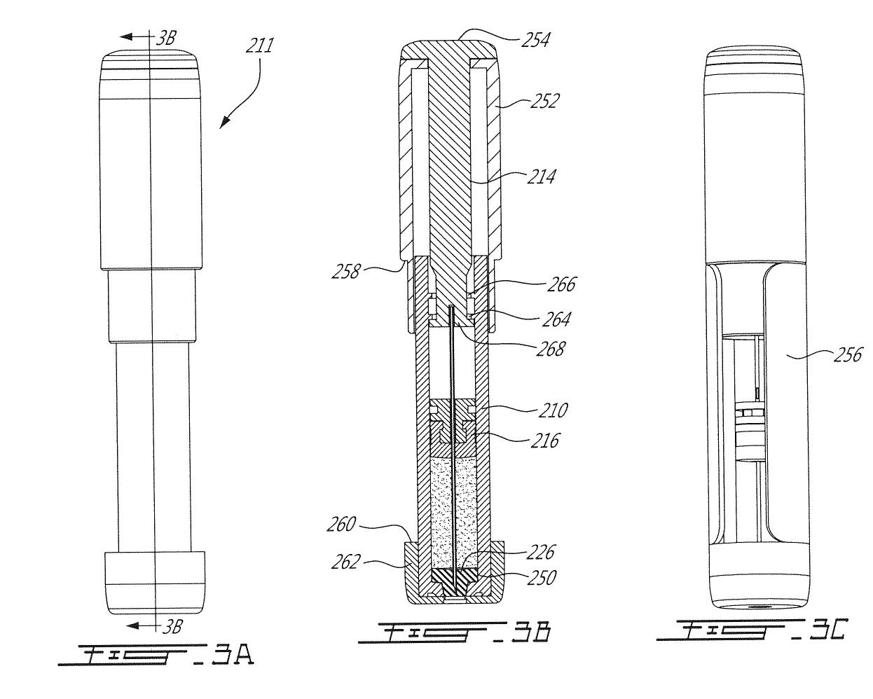

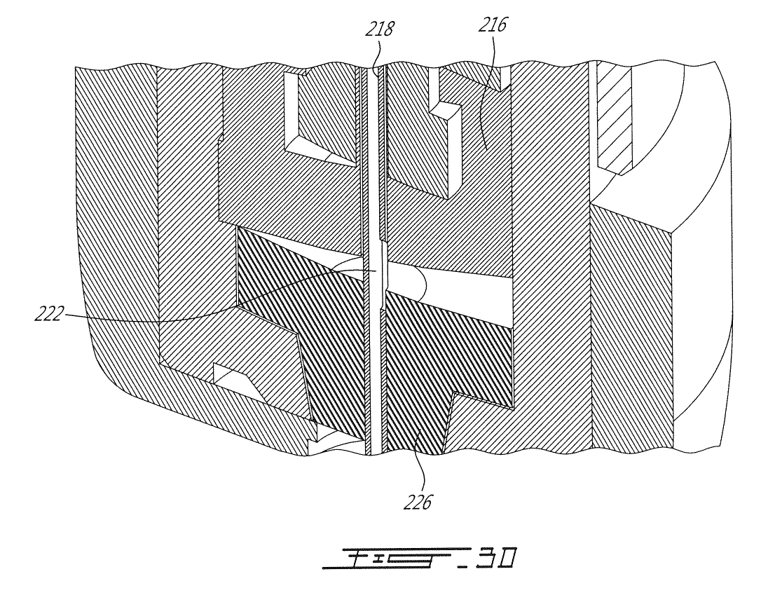

An example of a design adapted for intra-muscular injection is shown in FIGS. 3A, 3B and 3C. Referring to FIG. 3A, similar features to those shown in previously described designs will be recognized. Several differences can also be observed. For instance, the septum 226 is provided here in a thicker form and in a shape which snugly fits and abuts against a neck 250 provided in the bottom of a plastic shell of the barrel 210. The plunger body 214 also has a peripheral wall 252 connected to its tip 254. When the syringe 211 is in the initial position, such as shown in FIGS. 3A and 3B, a stop 256, which can be provided in the form of a transversally snapping collar for instance, can be provided around the barrel 210, to prevent accidental puncturing of the septum 226 should pressure be applied to the plunger body 214 without the intention of injecting. More specifically, the stop 256 is engaged between corresponding features 258, 260 provided in the peripheral wall of the plunger body 214 and a footer 262 of the barrel 210, respectively. To proceed with injection, the stop 256 is removed by pulling it transversally. FIG. 3B shows the syringe with the stop 256 removed, ready for injection. This embodiment also features two annular ribs 264, 266 in the upper portion of the barrel 210, between which the head 268 of the plunger body 214 can be retracted and trapped after the injection operation. Indeed, the lower rib 264, the head 268, or both, can be made of a resilient material to provide a snapping function. FIG. 3D shows the plunger seal 216 approaching the septum 226 and nearing the end of the injection. The aperture 222 in the needle 218 can be seen to extend across the remaining gap between the plunger seal 216 and the septum 226, allowing injection of the medical fluid all the way until the plunger seal 216 snugly engages the septum 226.

It will be understood that embodiments of the syringe can be provide with an automatically retracting feature which biases the plunger body to the retracted position in a manner that once the injection has been completed, the plunger body can be activated to move to its fully retracted position in the absence of an external force (e.g. when the volume-confining force applied by the user to proceed with injection has been discontinued).

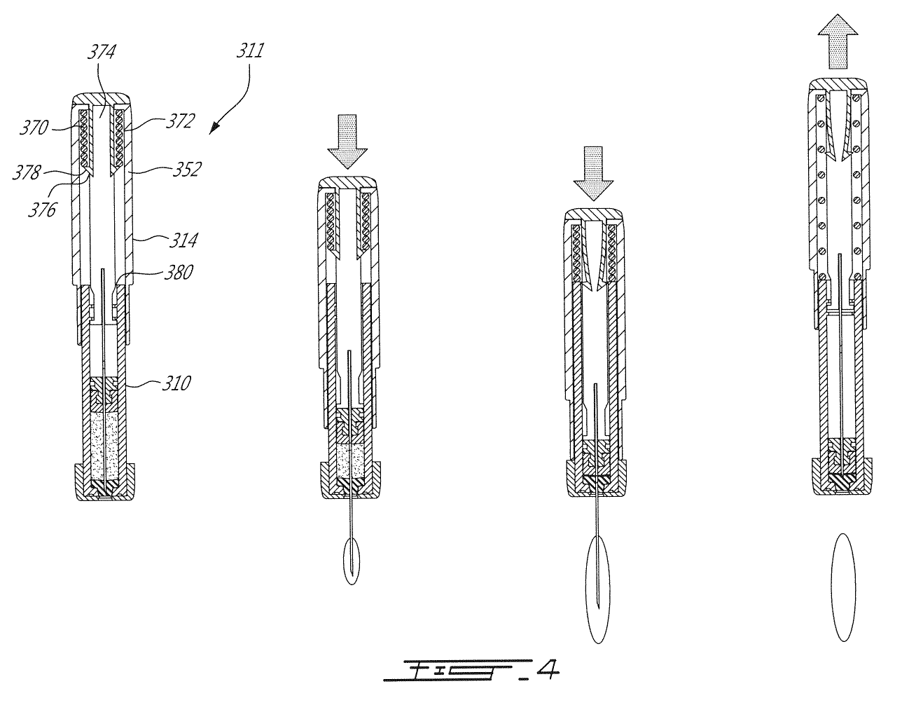

An example of a syringe 311 having an automatic retraction feature is provided in FIG. 4. More specifically, a compressed spring 370 is housed in an upper annular cavity 372 between a core 374 of the plunger body 314 and the peripheral wall 352. A mechanism is used to maintain the spring 370 in the compressed state until the injection has been completed. In this embodiment, the mechanism is provided in the form of retainer clasps 376 which project downwardly from the top of the plunger body 314 into the cavity 372. The retainer clasps have a sloping face 378 extending downwardly. The sloping faces 378 are designed to engage an upper edge 380 of the barrel 310 when the plunger body 314 has been fully pushed downwardly, in a manner that the barrel 310 reactively pushes the retainer clasps 376 inwardly, which frees the spring 370 from the retainer clasps 376. At this point, the retainer clasps 376 no longer retain the spring 370 in its compressed state and the spring 370 is free to extend, exerting a biasing force between the plunger body 314 and the barrel 310, which moves the plunger body 314 back into the fully retracted state in the absence of an external force, such as when the volume-confining force exerted by the user is discontinued.

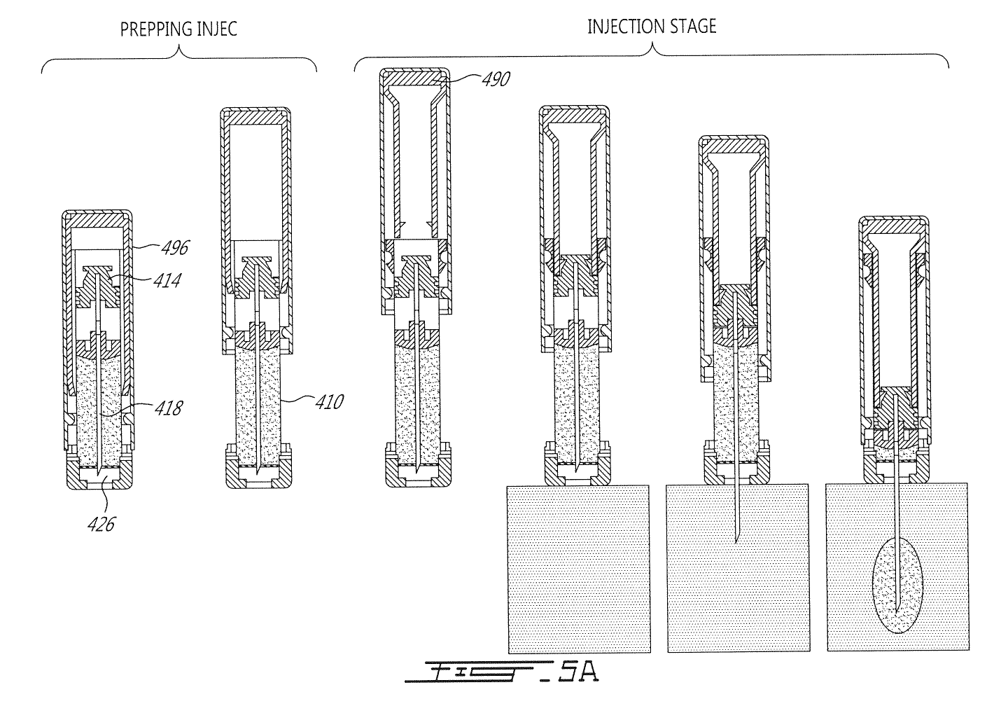

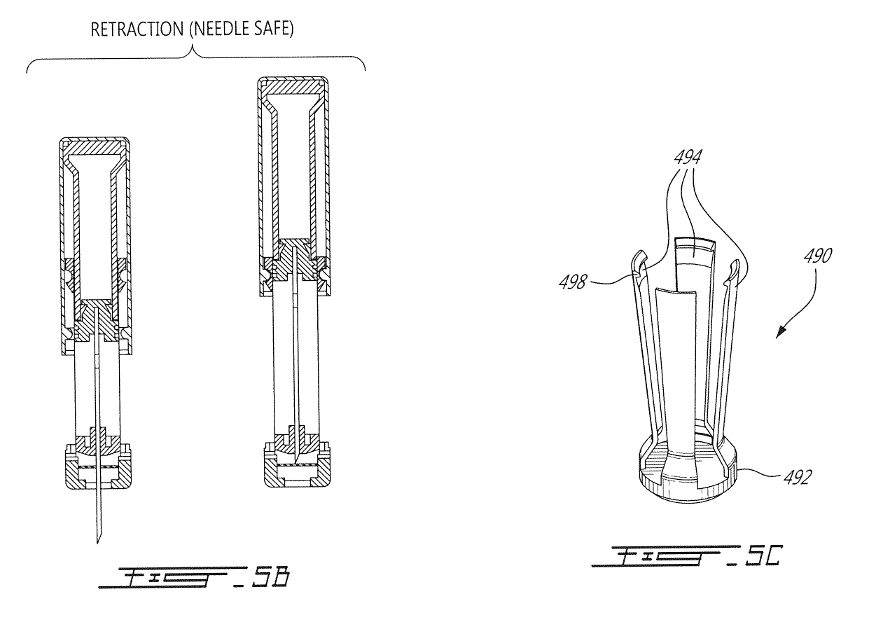

Still another example of a syringe is provided in FIGS. 5A and 5B. This embodiment is similar to the embodiment of FIG. 4. However, the plunger body has an engagement component 490 which has a shape shown in FIG. 5C, having a base 492 and circumferentially interspaced arms 494 projecting longitudinally from the base 492. The engagement component 490 is made of a resilient material. Initially, the arms 494 are moved outwardly and are positioned to surround the barrel 410. The engagement component 490 is housed within a cap 496. The cap 496 can be pushed downwardly around the barrel 410, such as shown on the left hand side of FIG. 5A. This initial configuration avoids unintentional activation of the needle 418 should a compressing force be unintentionally exerted between the cap and the barrel. To proceed with injection, the cap 496 is first pulled upwardly until the arms 492 are freed from engagement with the barrel 410. At that stage, the arms 494 revert to their original shape, and move radially inward. The arms 494 can then be pushed downwardly inside the barrel 410 by exerting a compressive force between the cap 496 and the barrel 410. The arms eventually engage the remainder of the plunger body 414 to which the needle 418 is anchored, at which point pushing the cap 496 downwardly will have the effect of pushing the needle 418 downwardly, puncturing the septum 426, continuing on the penetration span, and subsequently injecting the medical fluid. The arms 494 are provided with retaining clasps 498 at their tips which become engaged with the remainder of the plunger body 414, in a manner that once injection is terminated, the needle 418 can be retracted by pulling the cap 496 back up.

Pre-Clinical Trial

A preliminary pre-clinical trial was performed to assess the functionality a syringe which displaces the needle tip more deeply into the body as the medical fluid is being injected. More specifically, the trial was performed using a syringe such as shown in FIGS. 3A, 3B and 3C.

Two ˜10 kg Landrace Yorkshire Cross pigs received Intra Muscular (IM) injections of 500 μL of India ink in marked locations. Three injections were performed with a standard 1 cc tuberculin syringe (AIM) and 3 injections were performed with a syringe with a retractable needle which was fixedly mounted to the plunger (BIM). One animal was sacrificed at an early (E) time point (t=1 hour), the other was sacrificed at a later (L) time point (t=5 hours). The injections sites were evaluated on a standard scoring system with high quality digital pictures taken at a fixed distance for digital analysis. The injection sites were then excised for histological evaluation. The external injection site evaluation demonstrated that all injection sites were normal. Table 1, below, presents the mean summary data sheet for the excised injection site evaluation.

TABLE 1
Mean summary data sheet

| Injection Site | Degree | Intensity | Mean Volume (mm3) | ±SD |
|---|---|---|---|---|
| A-IM-L | 3.00 | 3.00 | 9,754.04 | 5,529.86 |
| B-IM-L | 3.00 | 3.00 | 15,970.25 | 4,037.75 |
| A-IM-E | 3.00 | 3.00 | 5,792.70 | 3,784.87 |
| B-IM-E | 2.00 | 2.00 | 6,456.94 | 5,948.85 |

In the test sample sacrificed at the later time point, the mean volume of injection was significantly higher for the syringe with the retractable needle than for the standard 1 cc tuberculin syringe.

In this description, specific embodiments are described with reference to associated figures for the purpose of providing example ways of embodying the invention(s). The invention(s) is/are not to be construed as being limited in scope to the specific embodiments described.

What is claimed is:

1. A syringe having a plunger body and a plunger seal both independently slidingly engaged within a cavity of a barrel for movement along a common longitudinal axis, and a needle anchored to the plunger body and extending across the plunger seal, the needle having an inlet aperture allowing fluid flow communication into an internal conduit of the needle, the barrel having a reservoir for receiving a medical fluid, the reservoir extending between the plunger seal and a bottom, the bottom being closed by a septum, wherein to proceed with injection, the plunger body is slid toward the plunger seal along a penetration span, during which movement the needle punctures the septum and protrudes from the bottom without injecting medical fluid, until the plunger body engages the plunger seal, after which the plunger body and the plunger seal are collectively moved to inject the medical fluid until the plunger seal reaches the bottom.

2. The syringe of claim 1 wherein the inlet aperture of the needle is a lateral aperture, and the lateral aperture becomes exposed to the medical fluid in the reservoir when the plunger body engages the plunger seal.

3. The syringe of claim 2 wherein the lateral aperture has a given length and a given position along the needle which are selected in a manner for the lateral aperture to be aligned with an inner face of the septum when the plunger seal has been moved into snug abutment with the bottom.

4. The syringe of claim 2 wherein the plunger seal has a cylindrical recess surrounding the needle and in communication with the reservoir, the cylindrical recess surrounding the lateral aperture when the lateral aperture is longitudinally aligned with the plunger seal.

5. The syringe of claim 1 wherein the barrel further comprises a projecting neck projecting from the bottom opposite the reservoir, the projecting neck being adapted to form a puckering of the skin when engaged thereagainst.

6. The syringe of claim 1 wherein the cavity of the barrel further comprises a first mating feature, and the plunger body further comprises a second mating feature, the first and second mating features becoming snappingly engaged with one another when the plunger body has been moved in a manner to fully retract the needle.

7. The syringe of claim 1 wherein the plunger body further comprises a peripheral wall slidingly engaged with an outer face of the barrel.

8. The syringe of claim 7 further comprising a stop snappingly engaged around the barrel, trapped between the peripheral wall and a footer of the barrel, in a manner to prevent longitudinal activation of the needle unless the stop has been snappingly and transversally removed.

9. The syringe of claim 7 further comprising a spring trapped in a compressed state within the plunger body, internally to the peripheral wall, the spring being untrapped to exert an extending, longitudinal force between plunger body the barrel once the plunger body has been moved fully into an injection position, in a manner to retract the plunger body and the syringe.

10. The syringe of claim 7 wherein the peripheral has an engagement component stretched into an extended configuration surrounding the barrel prior to said injection, the engagement component being transversally retracted when the peripheral wall is slid into a fully deployed configuration, in a manner to become slidingly engageable within the barrel, and engageable with the plunger body to perform said injection when the peripheral wall is slid back into a fully retracted configuration.

11. The syringe of claim 10 wherein the engagement component has retaining clasps which secure the plunger body when engaged therewith, the engagement component further retracting the secured plunger body when the peripheral wall is slid back into the extended configuration.

12. A syringe having a plunger having a plunger seal slidingly engaged within a cavity of a barrel, and a needle anchored to the plunger, the barrel having a reservoir for receiving a medical fluid, the reservoir extending between the plunger seal and a bottom, the bottom being closed by a septum, the needle having a lateral aperture allowing fluid flow communication between the reservoir and an internal conduit of the needle, wherein to proceed with injection, the plunger seal and the anchored needle are collectively moved along an axis of the syringe toward the bottom to inject the medical fluid until the plunger seal reaches the bottom, wherein the lateral aperture of the needle extends along a given axial length immediately below the plunger seal in a manner remain exposed to the reservoir until the plunger seal has reached the bottom.

13. The syringe of claim 12 wherein the plunger seal has a cylindrical recess surrounding the needle and in communication with the reservoir, the cylindrical recess surrounding the lateral aperture when the lateral aperture is longitudinally aligned with the plunger seal.

14. The syringe of claim 12 wherein the barrel further comprises a projecting neck projecting from the bottom opposite the reservoir, the projecting neck being adapted to form a puckering of the skin when engaged thereagainst.

15. The syringe of claim 12 wherein the cavity of the barrel further comprises a first mating feature, and the plunger body further comprises a second mating feature, the first and second mating features becoming snappingly engaged with one another when the plunger body has been moved in a manner to fully retract the needle.

16. The syringe of claim 1 wherein the plunger body further comprises a peripheral wall slidingly engaged with an outer face of the barrel.

17. The syringe of claim 16 further comprising a stop snappingly engaged around the barrel, trapped between the peripheral wall and a footer of the barrel, in a manner to prevent longitudinal activation of the needle unless the stop has been snappingly and transversally removed.

18. The syringe of claim 16 further comprising a spring trapped in a compressed state within the plunger body, internally to the peripheral wall, the spring being untrapped to exert an extending, longitudinal force between plunger body the barrel once the plunger body has been moved fully into an injection position, in a manner to retract the plunger body and the syringe.

19. The syringe of claim 16 wherein the peripheral has an engagement component stretched into an extended configuration surrounding the barrel prior to said injection, the engagement component being transversally retracted when the peripheral wall is slid into a fully deployed configuration, in a manner to become slidingly engageable within the barrel, and engageable with the plunger body to perform said injection when the peripheral wall is slid back into a fully retracted configuration.

20. A syringe having a plunger anchor to which a needle is anchored and a plunger seal both slidingly engaged within a cavity of a barrel, and a plunger actuator having at least two elastic arms slidingly engaged around the barrel, the arms being biased to a radially inward position and prevented by the barrel, the barrel having a septum seal at a bottom and an open top, wherein to proceed with injection, the plunger actuator is pulled upwardly until the arms are freed from the barrel, at which points the arms are freed and move to the radially inward position, and the plunger actuator can be pushed downwardly to engage the arms into the barrel via the open top, until they engage the plunger anchor and become operable to push the needle across the septum.
